SEQUENCE LISTING (2 sequences)

SEQ ID NO: 1 - protein, 833 aa, Thermus aquaticus
MNSGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKE

SEQ ID NO: 2 - DNA, 2502 bp, Thermus aquaticus
atgaattcggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaaggggccttcgtgggctttgtgctttcccgcaaggagcccatgtgggccgatcttctggccctggccgccgccagggggggccgggtccaccgggcccccgagccttataaagccctcagggacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtga

(19) United States
(12) Patent Application Publication
(10) Pub. No.: US 2024/0167004 A1
(43) Pub. Date: May 23, 2024

(54) HIGHLY SPECIFIC TAQ DNA POLYMERASE VARIANT AND USE THEREOF IN GENOME EDITING AND GENE MUTATION DETECTION

(71) Applicants: SHANGHAI TURTLE TECHNOLOGY CO., LTD., Shanghai (CN); SHANDONG UNIVERSITY, Shandong (CN)

(72) Inventors: Qilai HUANG, Shandong (CN); Xiaodan LIU, Shandong (CN); Ping DU, Shandong (CN); Bo LI, Shandong (CN)

(73) Assignees: SHANGHAI TURTLE TECHNOLOGY CO., LTD., Shanghai (CN); SHANDONG UNIVERSITY, Shandong (CN)

(21) Appl. No.: 18/283,815

(22) PCT Filed: Jul. 15, 2021
(86) PCT No.: PCT/CN2021/106566

§ 371 Date: Sep. 25, 2023

(30) Foreign Application Priority Data
Mar. 25, 2021 (CN) 202110320668.4

(51) Int. Cl. C12N 9/12

(52) U.S. Cl. CPC C12N 9/1252; C12Y 207/07007

(57) ABSTRACT

Provided are a highly specific Taq DNA polymerase variant and the use thereof in genome editing and gene mutation detection. All polar amino acids, directly interacting with a primer/template complex, on a Taq enzyme are selected to be mutated one by one to obtain 40 Taq variants, and extensive random mutagenesis is performed on the basis of the sequences of the variants and the sequences of wild type Taq enzymes to create a Taq mutant library. Then, a series of Taq mutants with a high specificity are screened on a qPCR screening system by means of taking a genome editing indels plasmid as a template, wherein the Taq mutants exhibit great advantages in CRISPR/Cas9 editing efficiency evaluation and single-cell cloning genotyping.

### TECHNICAL FIELD

The present invention belongs to the field of biotechnology, and specifically relates to a highly specific Taq DNA polymerase variant and a use thereof in genome editing and gene mutation detection.

### DESCRIPTION OF RELATED ART

The disclosure of this background section is merely intended to increase the understanding of the overall background of the present invention and is not necessarily considered an admission or in any form to imply that the information constitutes prior art known to those of ordinary skill in the art.

The CRISPR-Cas9 system enables convenient genome editing at a specific site with a short stretch of guide RNA. It has been widely applied in functional genomics research and holds great potential for treating diseases involving genetic variation. There are three main types of intended genome modifications, including: error-prone non-homologous end joining (NHEJ) repair, which occurs due to double-strand breaks and results in random indel mutations; homology-directed repair (HDR) using a DNA template, which can lead to precise base changes through homologous recombination, or direct base editing to induce accurate base modifications; gene regulation through the recruitment of transcription factors or chromatin modifying factors. For genome editing applications, it is usually necessary to evaluate the editing efficiency of given CRISPR targets and, in some cases, to genotype the single-cell clones obtained. Several methods have been developed, including gene-editing frequency digital polymerase chain reaction (GEF-dPCR), genome editing test PCR (getPCR), and annealing at critical temperature PCR (ACT-PCR), which discriminate DNA modifications from the wild-type sequences during the PCR amplification step. However, the discriminatory ability of Taq polymerase or TaqMan probes towards DNA mutations is limited, and careful optimization of experiments is required to obtain more accurate results. Using modified fluorescent probes or enhanced DNA polymerase variants with better mismatch discrimination capabilities than wild-type Taq polymerase can improve the accuracy of PCR detection. DNA polymerase variants can reliably detect genetic variations without the need for any probe or primer modifications, making them the most cost-effective strategy for improving the accuracy of gene variation detection.

The interactions between polymerase and the primer/template duplex at the minor groove are crucial for the assembly of the replication initiation complex. However, these interactions are highly redundant and exceed the minimum requirements for efficient DNA replication initiation. Substituting the involved amino acid residue can destroy the corresponding interactions and potentially lead to increased selectivity in the mismatch extension. Based on this principle, the reported rational evolution of DNA polymerase has mainly focused on substitutions of a few polar amino acids and basic amino acids in motif C. For example, highly selective Taq variants were identified through introducing functional mutations at 12 amino acid positions, and screening in a combinatorial library generated by molecular recombination. However, all these rational designs of DNA polymerase mutants were based on improving the selectivity of for 3′-terminal single nucleotide mismatches extension. The output of genome editing indels is largely unpredictable and variable, and the mismatch types between the PCR test primer and the indel-containing genomic DNA are extremely diverse. Therefore, a DNA polymerase variant with an improved ability to discriminate primer-template mismatches originating from gene modifications is greatly needed for genome editing studies. The enhanced Taq variant allows more accurate and convenient editing frequency determination and single-cell clone genotyping.

### SUMMARY

In response to the existing problems in current technologies, the present invention provides a highly specific Taq DNA polymerase variant and use thereof in genome editing and gene mutation detection. The present invention employs semi-rational directed molecular evolution to enhance the specificity of the wild-type full-length Taq DNA polymerase. All polar amino acids directly interacting with primer/template complexes on Taq enzyme were individually mutated, resulting in 40 Taq variants. Subsequently, extensive random mutagenesis was performed on these variants and the wild-type sequence to generate a Taq mutant library. Using qPCR screening system with genome editing indels plasmids as templates, a series of highly specific Taq mutants were identified, and exhibited significant advantages in CRISPR/Cas9 editing efficiency evaluation and single-cell clone genotyping, making it highly valuable for practical applications.

The specific invention involves the following technical solutions.

In a first aspect of the present invention, provides a variant of a Taq DNA polymerase, wherein including one or more mutation sites selected from a group consisting of S577A, W645R, I707V, R405Q, T569V, K354R, K531Q, L441M, S543A, R630W, F692Y, Y719F, M41, D371E, V518D, A798V, G32D, D238V, W398C, N485L, 1503F, R771K, E284K, 1614L, T588S, L789F, G59W, V155F, K508Q, R229G, E255V, Q489L, E90K, E132Q, P369T, T513A, D151G, S515A, R741Q, A294S, A675V, E688D, V740A, G173D, L5001, R37Q, T140S, D365N, T140A, L5381, P10A, E303G, L4841, R492M, F272S, E794D, E170G, K508T, D578L, E818V, 1799F, K206R, R229W, R249C, V390M, E404G, E267V, S577A, Q680H, R328M, R469C, E159D, D181H, P387L, A61T, D91N, K100E, K131N, A777V, P194H, P369T, T514V, Y719F, A118S, R435W, E708D, P6T, D177E, L252M, E465D, S699T, E135V, P316S, G422W, T385A, R137C, P685S, E818K, L828V, A414T, S515A, A600T, S361, E171K, S576A, E57D, D222Y, H28L, E112D, L245P, R630L, L351F, L657P, and P816S; wherein the mutation sites are numbered based on an amino acid sequence of wild-type Taq DNA polymerase as shown in SEQ ID NO: 1.

An amino acid sequence of the variant of the Taq DNA polymerase has at least 80% homology, more preferably, at least 90% homology, and most preferably, at least 95% homology, such as having at least 960%, 970%, 98%, 990% homology, compared to SEQ ID NO: 1.

The variant of the Taq DNA polymerase includes 1 to 6 mutation sites, more preferably 1 to 4 mutation sites, such as 1, 2, 3 or 4.

The variant of the Taq DNA polymerase is mutated from a wild-type Taq DNA polymerase shown in SEQ ID NO: 1, and is selected from the following variants:

| variant | |
|---|---|
| identifiers | mutated amino acid |
| Taq388 | S577A, W645R, I707V |
| Taq92 | R405Q, T569V |
| Taq99 | K354R, K531Q |
| Taq393 | L441M |
| Taq401 | S543A, R630W, F692Y, Y719F |
| Taq506 | M4I, D371E, V518D, A798V |
| Taq591 | G32D, D238V, W398C, N485L, I503F, R771K |
| Taq664 | E284K, I614L |
| Taq866 | T588S, L789F |
| Taq9 | G59W, V155F, K508Q |
| Taq1150 | R229G, E255V, Q489L |
| Taq1140 | E90K, E132Q, P369T, T513A |
| Taq761 | D151G, S515A, R741Q |
| Taq812 | A294S, A675V, E688D, V740A |
| Taq687 | G173D, L500I |
| Taq808 | R37Q, T140S, D365N |
| Taq1105 | T140A, L538I |
| Taq1151 | P10A, E303G, L484I, R492M |
| Taq1194 | F272S, E794D |
| Taq1108 | E170G, K508T, D578L, E818V |
| Taq1221 | I799F, K206R, R229W |
| Taq588 | R249C, V390M, E404G |
| Taq712 | E267V, S577A, Q680H |
| Taq1286 | R328M, R469C, |
| Taq1129 | E159D, D181H, P387L |
| Taq816 | A61T, D91N, K100E, K131N, A777V |
| Taq729 | P194H, P369T, T514V, Y719F |
| Taq1080 | A118S, R435W, E708D |
| Taq1312 | P6T, D177E, L252M, E465D, S699T |
| Taq1161 | E135V, P316S, G422W |
| Taq815 | T385A |
| Taq5 | R137C, P685S, E818K, L828V |
| Taq867 | A414T, S515A, A600T |
| Taq480 | S36I, E171K, S576A |
| Taq764 | E57D, D222Y |
| Taq926 | H28L, E112D |
| Taq903 | L245P |
| Taq1062 | R630L |
| Taq1201 | L351F, L657P, P816S |

The above-mentioned Taq DNA polymerase variants in the table are sorted in descending order according to their specificity. The top ten variants are considered excellent, as they exhibit at least 7 cycles higher Ct values compared to the wild-type Taq for the detection of indel mismatches. This indicates a significant improvement in their selectivity. Among these variants, Taq388 shows the highest selectivity, with an increase of approximately 23 cycles. Moreover, the Taq388 mutation leads to a highly significant enhancement in PCR selectivity for indel and single nucleotide variation mismatches. In practical applications, this Taq variant greatly improves the accuracy of genotyping single-cell clones using the getPCR method, making AS-qPCR SNP genotyping a more viable approach.

In a second aspect of the present invention, provided a polynucleotide molecule encoding a variant of a Taq DNA polymerase of the first aspect of the present invention described above.

In a third aspect of the present invention, provided a recombinant expression vector including a polynucleotide molecule of the second aspect of the present invention described above.

Specifically, the recombinant expression vector is obtained by effectively linking the above-mentioned multiple nucleotide molecules to an expression vector. The expression vector can be any one or a combination of a viral vector, plasmid, phage, phagemid, cosmid, fosmid or artificial chromosome; the virus vector may include an adenovirus vector, retrovirus vector or adeno-associated virus vector, and the artificial chromosome includes a bacterial artificial chromosome (BAC), a vector derived from phage P1 (PAC), a yeast artificial chromosome (YAC) or a mammalian artificial chromosome (MAC).

In a fourth aspect of the present invention, provided a host cell including a recombinant expression vector of the third aspect of the present invention described above or having a polynucleotide molecule of the second aspect of the present invention described above.

The host cell is prokaryotic cell or eukaryotic cell.

Specifically, the host cells can be any one or a combination of bacterial cells, fungal cells, or plant cells.

Wherein, the bacterial cells can be selected from any species within the genera of Escherichia, Agrobacterium, Bacillus, Streptomyces, Pseudomonas and Staphylococcus.

More specifically, the bacterial cells are Escherichia coli (such as E. coli DH5a), Agrobacterium tumefaciens (such as GV3101), Agrobacterium rhizogenes, Lactobacillus lactis, Bacillus subtilis, Bacillus cereus, or Pseudomonas fluorescens.

The fungal cells include yeast.

The plant cells can be transgenic plant cells, wherein the transgenic plants include Arabidopsis thaliana strains, corn strains, sorghum strains, potato strains, tomato strains, wheat strains, canola strains, rapeseed strains, soybean strains, rice strains, barley strains, or tobacco strains.

In a fifth aspect of the present invention, provided a method for preparing a variant of a Taq DNA polymerase of the first aspect of the present invention described above, including: culturing a host cell of the fourth aspect of the present invention described above, thereby expressing the variant; and isolating the variant.

In a sixth aspect of the present invention, provided a kit including a variant of a Taq DNA polymerase of the first aspect of the present invention described above.

In a seventh aspect of the present invention, provided an application of a variant of a Taq DNA polymerase of the first aspect of the present invention described above, a polynucleotide molecule of the second aspect of the present invention described above, a recombinant expression vector of the third aspect of the present invention described above, a host cell of the fourth aspect of the present invention described above, or a kit of the sixth aspect of the present invention described above, in any one or more of the following:
1) genome editing detection (such as CRISPR/Cas9-based genome editing); and
2) gene mutation detection (such as single cell clone genotyping and SNP genotyping analysis).

Beneficial Technical Effects of the above technical solution(s):

The above technical solutions provide highly specific Taq DNA polymerase variant and use thereof in genome editing and gene mutation detection. The present invention employs semi-rational directed molecular evolution to enhance the specificity of the wild-type full-length Taq DNA polymerase. All polar amino acids directly interacting with primer/template complexes on Taq enzyme were individually mutated, resulting in 40 Taq variants. Subsequently, extensive random mutagenesis was performed on these variants and the wild-type sequence to generate a Taq mutant library. Using qPCR screening system with genome editing indels plasmids as templates, a series of highly specific Taq mutants were identified. Among them, the variant Taq388, which had three amino acid mutations (S577A in the palm region, W645R, and I707V in the fingers region), exhibited significant advantages in CRISPR/Cas9 editing efficiency evaluation and single-cell clone genotyping. Additionally, this variant also demonstrated excellent performance in detecting naturally occurring genetic variations such as SNPs, making it highly valuable for practical applications.

### BRIEF DESCRIPTION OF THE DRAWINGS

The drawings accompanying the specification forming a part of the present invention serve to provide a further understanding of the present invention. The schematic embodiments of the present invention and their description are used to explain the present invention and do not constitute an improper limitation of the present invention.

FIG. 1. Illustration of the strategy for directed evolution of high-specific Taq according to the present invention.

(a) Schematic diagram of the 40 polar amino acids directly interacting with primer/template duplex. The polar amino acids are indicated by arrows in the sequence. (b) The principle and flowchart of directed Taq evolution. The Taq mutagenesis library contains random mutagenesis based on the 40 individual variants. The screening system uses 26 plasmid constructs as a template, each harboring a mimic indel at the HOXB13 gene sgRNA target 1. The test primer anneals to the wild sequence at the editing region and creates mismatches at the 3′ end when annealed to the indel templates. A control amplification on the neighboring region is included to reflect polymerase activity. Highly selective Taq variants are those with higher test amplicon Ct values compared to the wild-type Taq.

FIG. 2. Screening and structure analysis of improved Taq variants according to the present invention.

(a) Evaluation of the 40 Taq variants for the polymerase activity and ability in discriminating indel-derived mismatches by using colonies grown in a lysogeny broth (LB) agar plate containing IPTG. A Ct value of 45 represents no polymerase activity remaining. Mean±S.E.M (standard error of mean), n=3 technical replicates. (b) Polymerase activity and indel sensitivity evaluation for 1,316 transformants from the random mutation libraries in the initial round of screening. The 176 transformants having full polymerase activity, with increased specificity, and highlighted. (c) Further polymerase activity and indel sensitivity evaluation for the 176 transformants. Thirty-nine transformants confirmed for their increased indel sensitivity are highlighted. (d) Characterization of the 39 purified Taq variants. The three variants with the best specificity are indicated with arrows.

FIG. 3. Analysis of the selective amplification ability of Taq388 of the present invention on indel variations according to the present invention.

(a) In the TaqMan probe-based qPCR system, the selective evaluation of Taq388 on primer-template mismatches caused by simulated indel mutations in the HOXB13 gene mixture in qPCR reactions. (b) Evaluation of the ability of Taq388 to identify and selectively amplify the above indels in the SYBR Green qPCR system.

FIG. 4. Ability of Taq388 of the present invention to discriminate single-nucleotide mismatches.

(a) Evaluation of Taq variant for sensitivity to primer-template mismatches located at the last nucleotide at the primer 3′ end. The primers and templates are illustrated. The relative PCR signals were calculated with the matched template at 100%. Mean±S.E.M, n=3 independent technical replicates. (b) Evaluation of the Taq variant for sensitivity with primer-template mismatches located at the penultimate nucleotide at the primer 3′ end. Mean±S.E.M, n=3 independent technical replicates. (c-d) The ability of Taq388 to discriminate different alleles of the breast cancer risk SNP rs4808611 in the allele-specific qPCR analysis of genomic DNA from MCF7 (C/C) (c) and T-47D (T/T) (d).

FIG. 5. Application of Taq388 of the present invention in genome editing detection by getPCR.

(a-b) Comparison of the Taq388 and wild-type Taq in recognizing the 26 individual indels on the HOXB13 gene. Plasmids bearing each indel can be detected using TaqMan probe method (a) or SYBR Green method (b). (c) Comparison between Taq388 and the wild-type Taq in the genotyping of the Lenti-X 293 T single-cell colonies that underwent genome editing at sgRNA targeting site 2 of HOXB13 gene. All 20 clones contain double-allele indel mutations, as previously determined. (d) Comparison of Taq388 with the wild-type Taq in genotyping of Lenti-X 293 T single-cell colonies edited at sgRNA targeting site 1 of DYRK1A gene. All edited clones were double allele indel variants, as confirmed by Sanger sequencing. Watching bases in the detection primer were highlighted, and the PAM sequence “NGG” is shown in light color. The larger the Ct value, the better the indel-mismatch sensitivity of the enzyme. A Ct value of 45 indicates no amplification signal. (Mean±S.E.M, n=3 independent technical replicates).

FIG. 6. Application of Taq388 of the present invention in SNP genotyping.

(a-e) Application of Taq388 in genotyping of 30 genomic DNA samples for the five SNP sites, rs2236007 (a), rs4808611 (b), rs11055880 (c), rs2290203 (d), and rs2046210 (e), with wild-type Taq included for comparison. The ratio of each allele was calculated using the formula: allele 1%=2^{-Ct(allele1)}/[2^{-Ct(allele1)}+2^{-Ct(allele2)}]. The points on the axis are homozygous, and the points between the axis are heterozygous. Taq388 successfully discriminated each genotype, while wild-type Taq could not determine the genotypes due to poor specificity. (f-j) Scatter plot of the end-point fluorescence of Taq388 and wild-type Taq for the allele-specific qPCR analysis of the five SNPs. The gray dots near the origin are the no-template amplification samples used as controls.

FIG. 7. Evolution of high-specific Taq of the present invention.

(a) Amino acid mutations of the 39 Taq variants determined by Sanger sequencing, with shaded clones representing the top 10 variants with the best selectivity. (b) SDS-PAGE analysis of the 39 Taq variants expressed and purified from E. coli. (c) The mutation frequency for the wild-type Taq and Taq388 during PCR amplification, determined by Sanger sequencing analysis. The Taq coding sequences amplified from the Taq388 variants were cloned into plasmids, and 20 single-cell clones of each Taq variant were sequenced to identify mutations. (d) Mutagenesis types generated in the PCR amplification using Taq388 and wild-type Taq.

FIG. 8. Sensitivity of Taq388 of the present invention to mismatch.

(a-c) The ability of Taq388 to discriminate different alleles of breast cancer risk SNP rs2236007 in allele-specific qPCR analysis on genomic DNA from T-47D (G/G) and VCaP cells (A/A), and Sanger sequencing analysis of the rs2236007 genotype in the two cancer cell lines. (d) The ability of Taq388 to discriminate indels was compared with five commercially available qPCR detection pre-mix products as indicated in the figure; the ability of Taq388 to discriminate SNP alleles of rs2236007 was compared with five commercial qPCR master mixes indicated in the figure.

FIG. 9. Comparison of Taq388 of the present invention with other strategies to enhance PCR selectivity in SNP detection.

(a) Genetic variation detection of TP53-G818A in the SW620 genomic DNA through AS-qPCR. Taq388 was compared with the blocking primer with ddC at the 3′ end. (b) TP53-G839A variation detection in the MDA-MB-231 genomic DNA through AS-qPCR. Taq388 was compared with the blocking primer with ddC at the 3′ end. (c) TP53-G818A variation detection in SW620 genomic DNA through AS-qPCR. Taq388 was compared with the primer having LNA at the 3′ end. (d) TP53-G839A detection using MDA-MB-231 genomic DNA with AS-qPCR. Taq388 was compared with the LNA primer. (e) TP53-G839A from MDA-MB-231 cells through qPCR. Taq388 was compared with the blocking primer with phosphorylated 3′ end.

FIG. 10. The evaluation of wild-type Taq in endpoint SNP genotyping according to the present invention.

(a-e) Sanger sequencing chromatography of the seven DNA samples that exhibited various allele ratios in the qPCR analysis for the five SNP sites. The locations of the five SNPs are highlighted in dark background. The Sanger sequencing results are highly consistent with the qPCR results.

### DESCRIPTION OF THE EMBODIMENTS

It should be noted that the following detailed descriptions are exemplary and intended to provide further explanation of the present invention. Unless otherwise indicated, all techniques and scientific terminology used in this document have the same meaning as understood by those skilled in the art to which the present invention belongs.

It should be noted that the terms used herein are only for describing specific embodiments, and not intended to limit the exemplary embodiments according to the present application. As used herein, unless the context otherwise clearly indicates, the singular forms also intend to include the plural forms, and it should also be understood that when the term “comprising” and/or “including” is used in this specification, it indicates the presence of features, steps, operations, devices, components and/or combinations thereof. The experimental methods without specific conditions in the following specific embodiments are generally carried out according to the conventional methods and conditions of molecular biology in this field of technology, which are fully explained in the literature. See for example the techniques and conditions described by Sambrook et al., “Molecular Cloning: A Laboratory Manual”, or according to the conditions suggested by the manufacturer.

The following examples further explain and illustrate the invention, but do not limit the scope of the invention.

### EXAMPLES

1. Experimental Materials and Methods

1.1 Site-Directed and Random Mutagenesis of Taq Polymerase

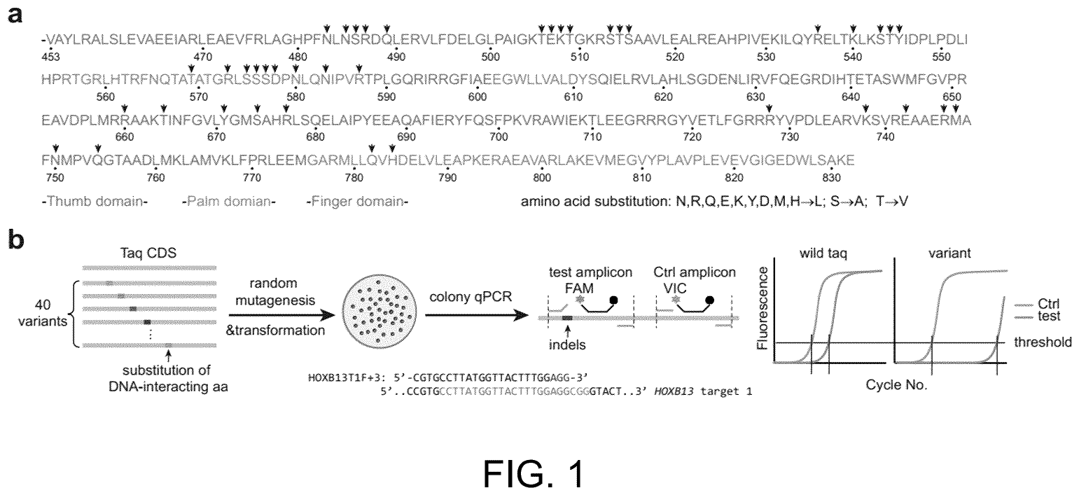

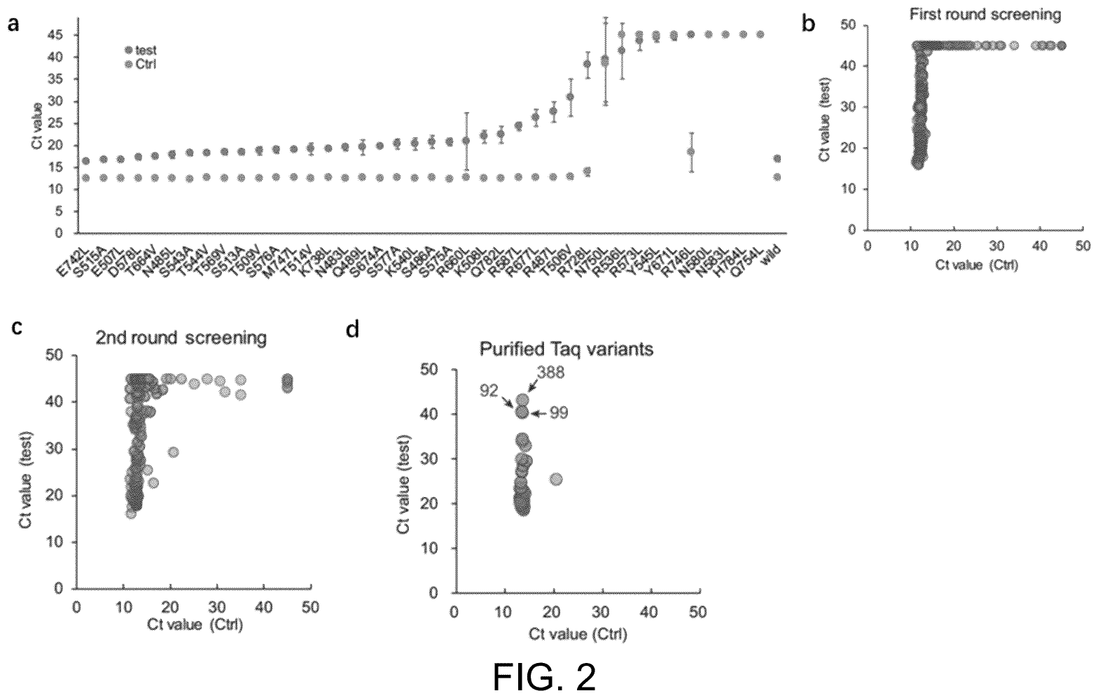

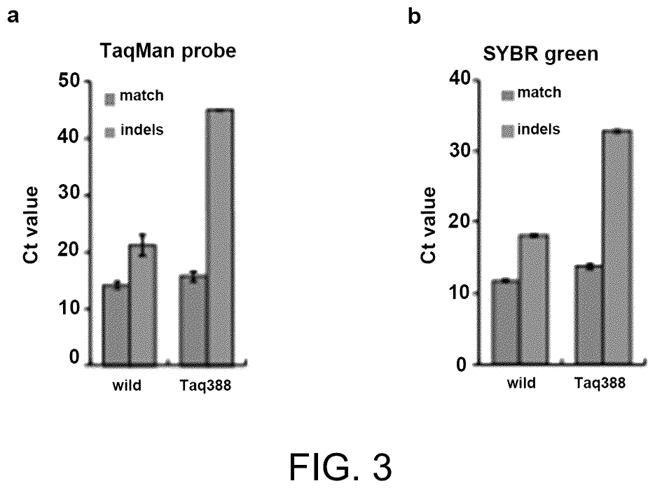

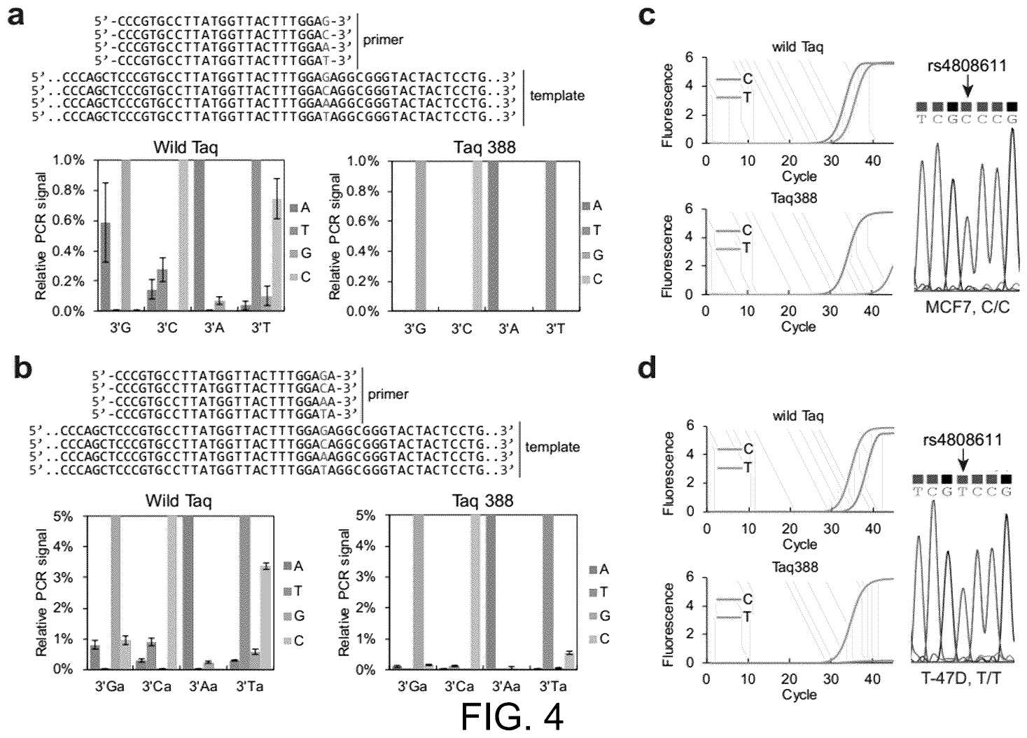

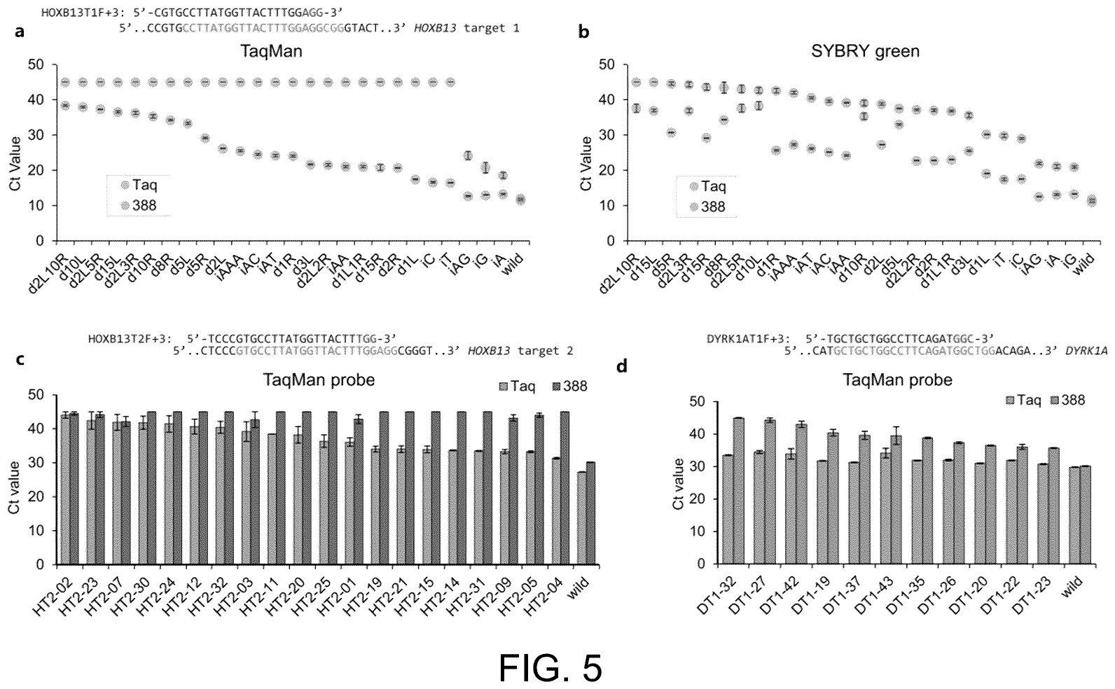

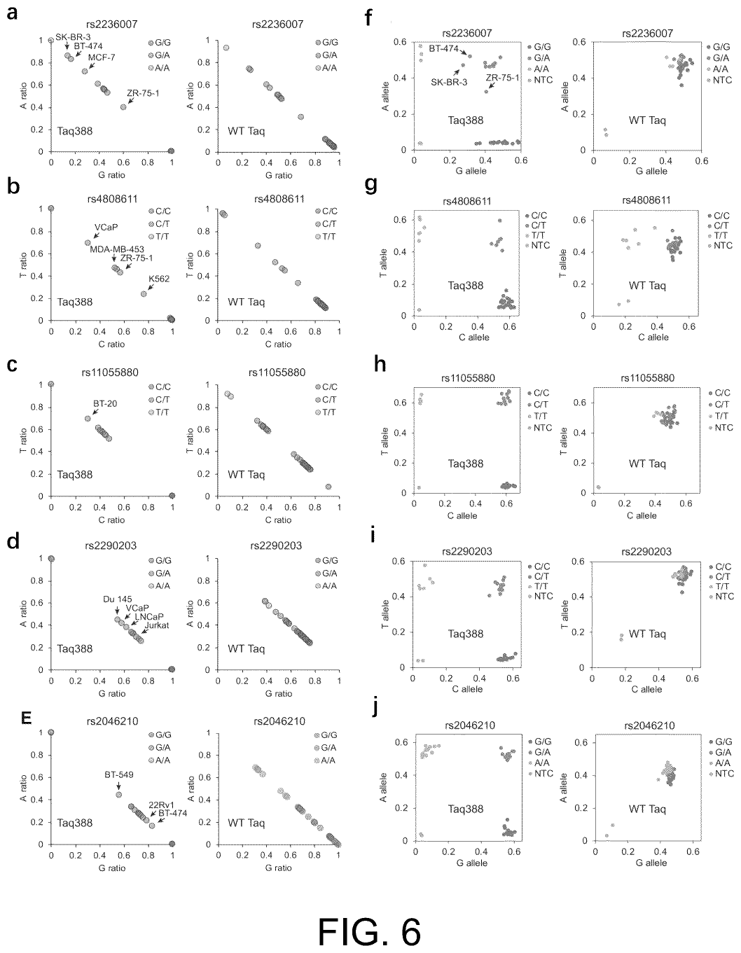

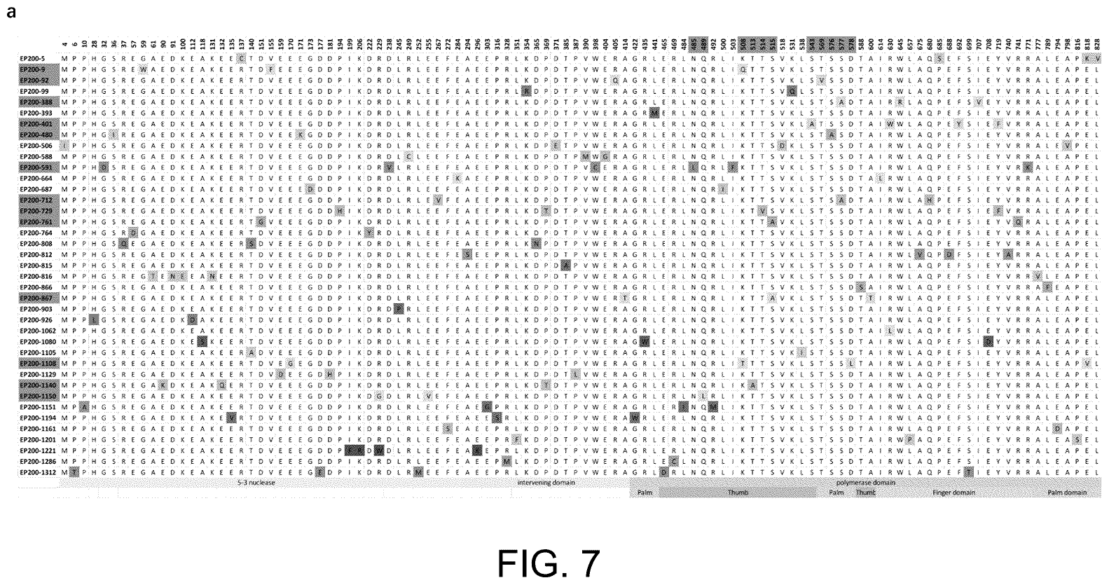

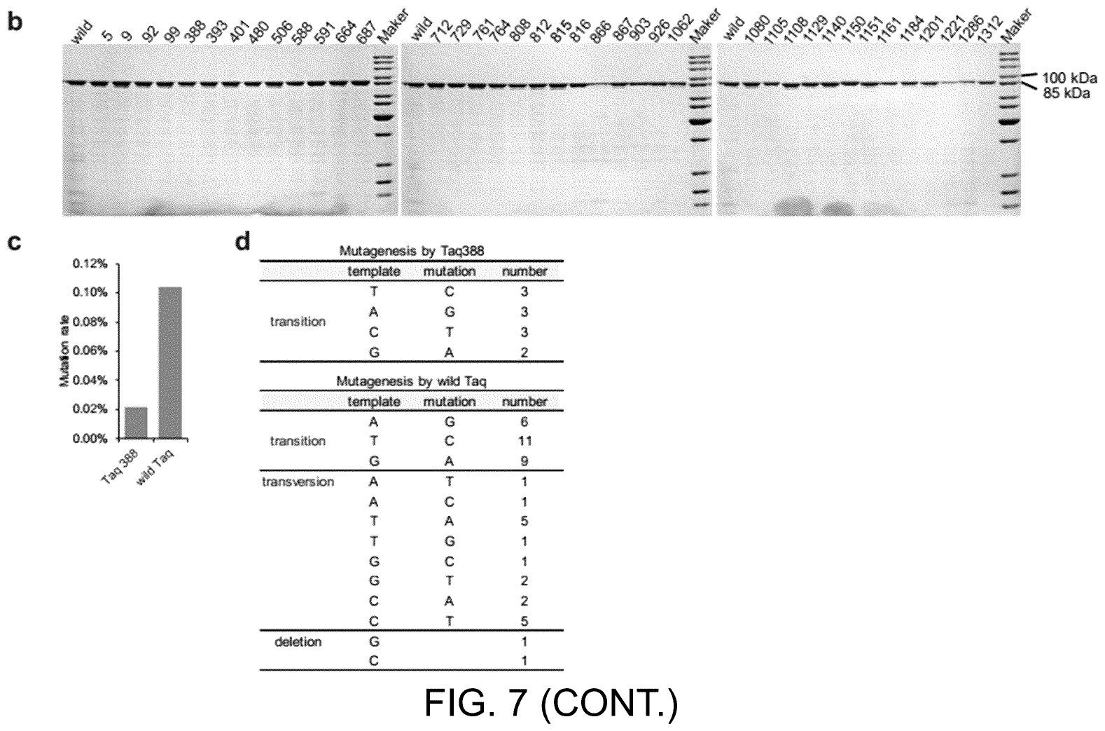

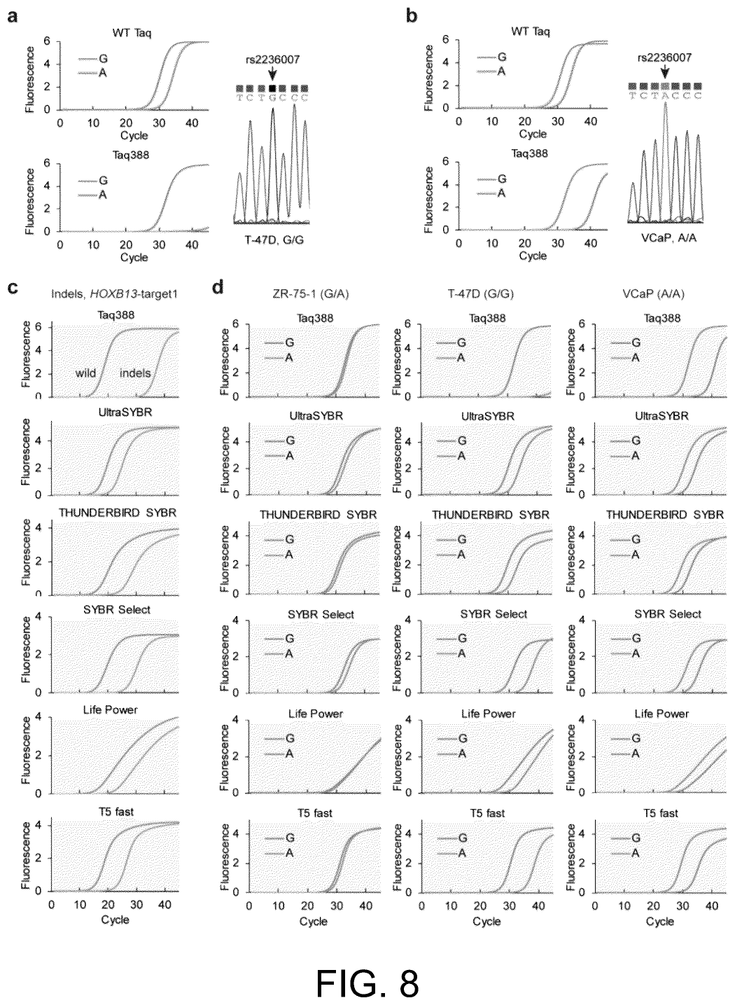

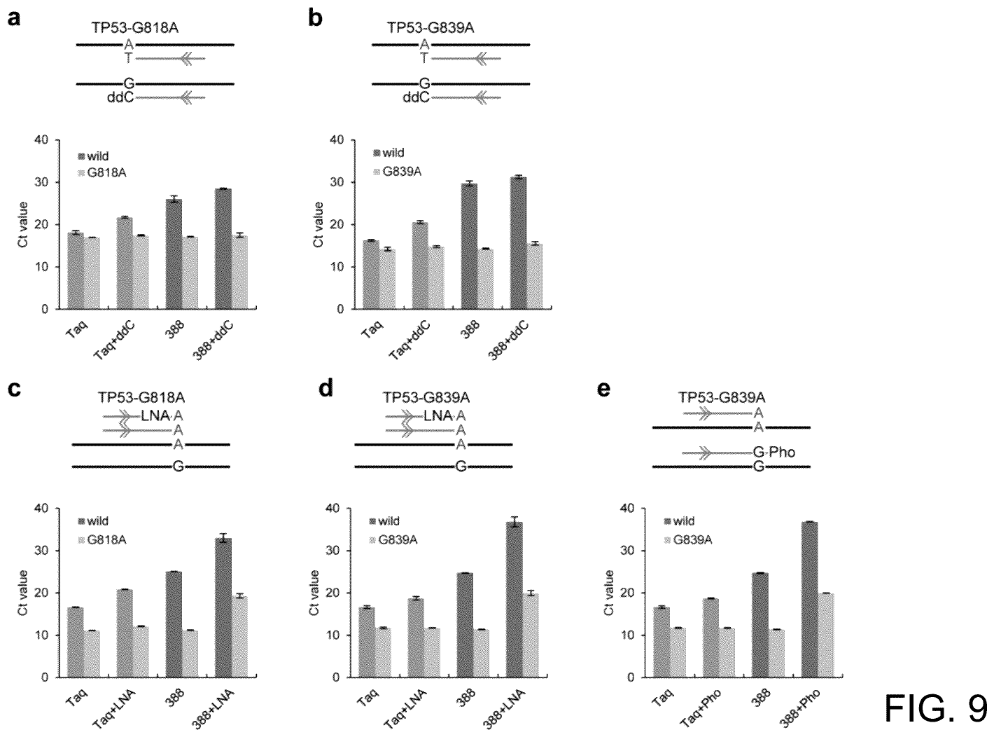

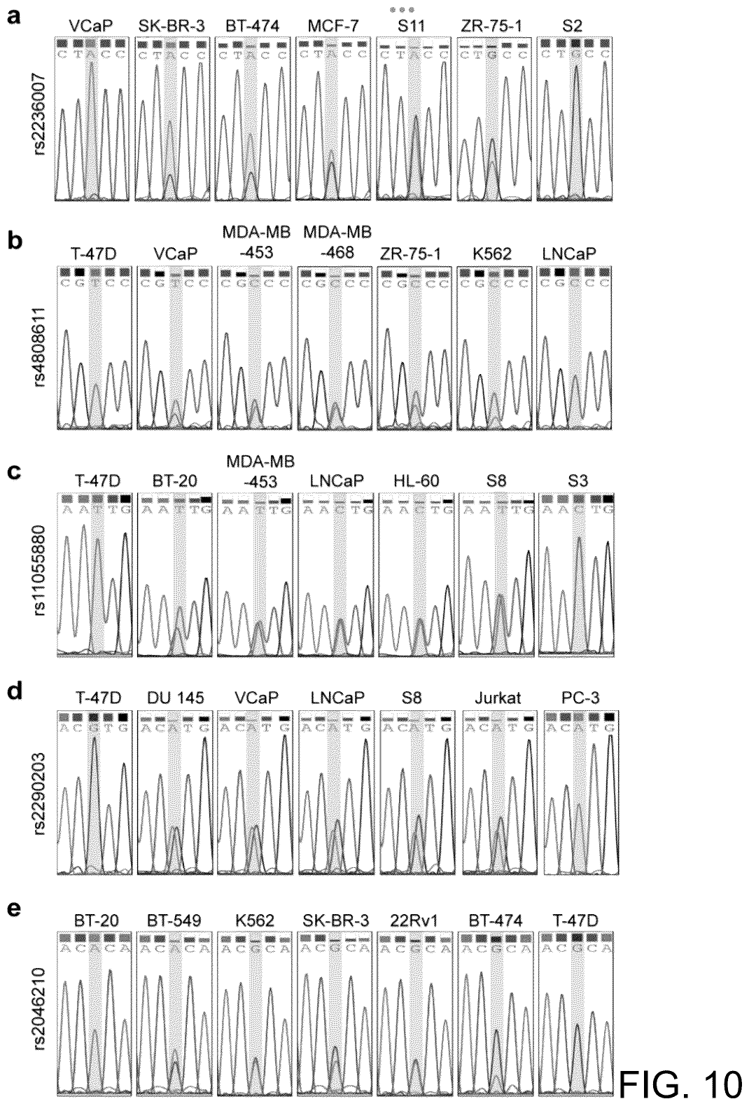

The plasmid pAKTaq (Addgene #25712) used for bacterial expression of Taq polymerase was purchased from the Addgene website. Site-directed mutagenesis PCR was performed on pAKTaq to replace each of the 40 polar amino acids involved in Taq enzyme-DNA interactions (a of FIG. 1). The site-directed mutagenesis PCR reaction was carried out in a 20 μL volume containing 4 pmol of site-directed mutagenesis primers and 10 μL of 2× Prime STAR Max Premix (TaKaRa). The PCR program consisted of an initial denaturation at 98° C. for 15 seconds, followed by 25 cycles of denaturation at 98° C. for 10 seconds, extension at 72° C. for 2 minutes, and a final extension at 72° C. for 5 minutes. The PCR products were treated with FastDigest DpnI (Thermo Fisher Scientific) at 37° C. for 2 hours and then used directly for transformation into DH5a competent cells. The transformed cells were plated on LB agar plates containing ampicillin and incubated overnight with inversion at 37° C. in the incubator. The next day, single colonies were picked and inoculated into LB medium and grown overnight at 37° C. with shaking at 250 rpm. Plasmid DNA was extracted and used for Sanger sequencing.

The 40 confirmed mutant variants were mixed in equal proportions and combined with pAKTaq in a 1:1 ratio. This mixture was then used as a template for random mutagenesis using the GeneMorph II Random Mutagenesis Kit (Agilent Technologies) through error-prone PCR. The error-prone PCR reaction was performed in a 25 μL reaction system containing 2.5 μL of 10× Mutazyme II reaction buffer, 0.5 μL of 40 mM dNTP mix, 1 pmol of upstream and downstream primers, 0.5 μL of Mutazyme II DNA polymerase (2.5 U/μL), and 15 ng of template plasmid. The PCR program consisted of an initial denaturation at 95° C. for 2 minutes, followed by 10 cycles of denaturation at 95° C. for 30 seconds, annealing at 60° C. for 30 seconds, and extension at 72° C. for 3 minutes, with a final extension at 72° C. for 10 minutes. The PCR products were then cloned into the original expression vector using EcoRI/SalI double digestion. The frequency of mutations in the transformed clones was determined by single-cloning Sanger sequencing. The template amount and cycle number of error-prone PCR were adjusted according to the product manual, until the desired mutation frequency was achieved that met requirements.

1.2 Colony qPCR Screening for High-Specificity Taq Variants

The random mutagenesis library plasmid was transformed into E. coli DH5a competent cells, and the expression of Taq variants was induced by growing the cells on LB solid medium containing ampicillin and IPTG. To determine the activity and specificity of different Taq variants, 26 pcDNA3.1-based plasmids carrying simulated CRISPR/Cas9 gene-editing indels in the HOXB13 gene were used as PCR templates for screening using colony-based quantitative PCR (qPCR) method. The single tube qPCR reaction included two amplicons, namely the target amplicon and the control amplicon. The upstream primer of the target amplicon spanned the simulated genomic editing site, which allowed evaluation of Taq enzyme's selectivity for primer-template mismatches caused by indels. The target amplicon was detected using a FAM-labeled TaqMan probe. The control amplicon matched the adjacent sequence where no mutation occurred and served as a measure of Taq variant's polymerase activity. It was detected using a VIC-labeled TaqMan probe. All primers used were designed based on the getPCR strategy. It is worth noting that the plasmid was linearized using Fast Digest NotI (Thermo Scientific™, CAT #FD0593) to avoid fluorescence signal interference between the two probes. Single colonies expressing Taq variants grown on LB agar plates containing IPTG were picked, and 10 μL of 1× Taq enzyme screening buffer (50 mM Tris-HCl [pH 8.8], 16 mM [NH₄]₂SO4, 0.1% [v/v] Tween®20, 2.5 μM MgCl₂, 0.25 mM each dNTP) was added and mixed well. Then, 7 μL to 20 μL of the mixture was added to the qPCR system. The working concentrations of each primer and probe were 0.2 μM and 0.1 μM, respectively. The qPCR program was as follows: initial denaturation at 95° C. for 5 minutes, followed by 45 cycles of denaturation at 95° C. for 30 seconds, annealing at 68° C. for 30 seconds, and extension at 72° C. for 10 seconds. The desired Taq variant was expected to show an increase in Ct value for the target amplicon while the Ct value for the control amplicon remained unchanged, indicating increased specificity.

1.3 Purification of Taq Variants

After two rounds of colony qPCR screening, a total of 39 improved variants were obtained. Each variant's amino acid mutations were determined through Sanger sequencing analysis. These variants were then expressed and purified in E. coli. For each clone, the corresponding 100 μL overnight culture was transferred into 4 ml of LB liquid medium containing ampicillin resistance. The culture was activated at 37° C. and 250 rpm for approximately 4 hours until reaching an OD600 nm of 0.8. Protein expression was induced by adding 1 mM IPTG and the culture was incubated at 37° C. and 250 rpm for 12 hours. The bacterial cells were collected by centrifugation at 5000 rpm for 3 minutes. The pellet was resuspended in 400 μL of buffer (50 mM Tris-HCl [pH 7.9], 50 mM sucrose, 1 mM EDTA [pH 8.0]) and centrifuged again at 5000 rpm for 3 minutes at room temperature to collect bacterial cells. The bacterial cells were then incubated with 200 μL of pre-lysis solution (50 mM Tris-HCl [pH 7.9], 50 mM sucrose, 1 mM EDTA [pH 8.0], 4 mg/mL lysozyme [Amresco]) at room temperature for 15 minutes. Next, the cell suspension was frozen in a −80° C. freezer for 30 minutes and then thawed completely at room temperature. After one cycle of freezing and thawing, the solution was immediately incubated in a 37° C. water bath for 15 minutes. 1 μL of 5 mg/ml DNaseI, 1 μL of 1 M CaCl₂), and 2 μL of 1 M MnCl₂ were added and mixed well. The mixture was further incubated at 37° C. for 30 minutes. Then, 200 μL of lysis buffer (10 mM Tris-HCl [pH 7.9], 50 mM KCl, 1 mM EDTA [pH 8.0], 0.5% [v/v] Tween®20, 0.5% [v/v] NP40) was added and mixed well. The lysate was incubated at 75° C. for 1 hour and then centrifuged at 15000 rpm for 10 minutes at 4° C. to collect the supernatant. 0.12 g of solid (NH₄)₂SO₄ was added to the supernatant, followed by incubation at 4° C. for 30 minutes with rotation. The solution was then centrifuged at 15000 rpm for 20 minutes at 4° C. to collect the precipitate. It was resuspended in 300 μL of storage buffer (50 mM Tris-HCl [pH 7.9], 50 mM KCl, 0.1 mM EDTA [pH 8.0], 1× protease inhibitor, 0.1% [v/v] Tween®20, 50% [v/v] glycerol) and stored at −20° C.

Finally, the content of Taq variants in the protein samples was analyzed by SDS-PAGE electrophoresis. The protein samples were loaded into a gel composed of 12% separating gel and 5% stacking gel. After electrophoresis, the gel was stained with eStainTML1 protein stain (GenScript) and analyzed using the Quantum-ST5 imaging system (VILBER LOURMAT, France).

1.4 Amplification Fidelity Analysis of the Taq388 Mutant Variant

To analyze the fidelity of the Taq388 variant compared to wild-type Taq, the Taq polymerase coding sequence from plasmid pAKTaq was used as a template and performed PCR amplification using 10×Taq enzyme screening buffer. The PCR products were digested with FastDigest EcoRI (Thermo) and FastDigest SalI (Thermo), and then inserted into the same digested vector pAKTaq. The resulting ligation products were transformed into E. coli DH5a competent cells. Twenty individual clones were selected for Sanger sequencing to calculate the number of mutated bases in the amplicon sequence for each clone and determine the mutation frequency.

1.5 GetPCR Analysis Conditions

For the SYBR Green-based getPCR method, a 15 μL reaction mixture contained 7.5 μL of 2×Taq buffer, 3 pmol of each primer, 0.005 ng of plasmid DNA or 3 ng of genomic DNA as template, and 1 μL of Taq polymerase. The analysis was performed on a Rotor-Gene Q 2plex qPCR machine (Qiagen) with the following program: initial denaturation at 95° C. for 5 minutes, denaturation at 95° C. for 30 seconds, primer annealing at 64-70° C. for 30 seconds, extension at 72° C. for 10 seconds. For analysis on a LightCycler®96 thermal cycler (Roche Applied Science, Germany), the conditions were as follows: initial denaturation at 95° C. for 5 minutes.

For the TaqMan probe-based getPCR method, the reaction mixture was 20 μL, including 2 μL of 10×Taq enzyme screening buffer, 0.1 ng of plasmid DNA or 10 ng of genomic DNA as template, 4 pmol of primers, 2 pmol of probe, and 1 μL of Taq polymerase. Real-time PCR was performed on a qPCR machine (Rotor-Gene Q 2plex, Qiagen) with the following program: initial denaturation at 95° C. for 5 minutes, denaturation at 95° C. for 30 seconds, primer annealing at 64-70° C. for 30 seconds, extension at 72° C. for 10 seconds. When using the LightCycler®96 thermal cycler (Roche Applied Science, Germany), the conditions were: initial denaturation cycle (95° C., 5 minutes), followed by 45 PCR cycles (95° C., 15 seconds; 64-70° C., 15 seconds; 72° C., 15 seconds).

1.6 Selectivity Analysis of Taq388 in Indel Detection

The selectivity of Taq388 in detecting primer-template mismatches caused by indels was analyzed using the SYBR Green and TaqMan probe-based qPCR systems. The PCR templates used were 26 synthetic plasmids that mimic indels and were employed in the Taq variant screening system. When mixed together, these 26 plasmids simulate a mixture of indels generated by genome editing. Each plasmid, when used individually as a template, represents a single-cell clone with a homozygous indel isolated in genome editing experiments. For TaqMan probe-based qPCR detection, a 20 μL reaction mixture was used, consisting of one pair of detection primers and one corresponding TaqMan detection probe, as well as one pair of control primers and one control TaqMan probe. The SYBR Green method differs in that it does not use TaqMan probes and requires separate detection amplification and control amplification in two reaction tubes.

In the actual application of genome editing, the selectivity of Taq388 was evaluated using 31 lenti-X 293T single-cell clones with CRISPR/Cas9-mediated genome editing. Among these clones, 20 single-cell clones had biallelic gene editing in the HOXB13 gene, and 11 single-cell clones had biallelic gene editing in the DYRK1A gene. The genome of the Lenti-X 293T cell line without editing served as an internal reference for both series. The qPCR assays with SYBR Green or TaqMan probes were performed using a LightCycler®96 instrument (Roche) (c and d of FIG. 5). The PCR conditions and program were described in the getPCR analysis conditions section.

1.7 Application of Taq388 in SNP Genotyping

In the analysis, 30 genomic DNA samples were used, including 10 from breast cancer cell lines (MCF7, T47D, MDA-MB-231, BT-474, BT-20, BT-549, SK-BR-3, ZR-75-1, MDA-MB-468, MDA-MB-453), 5 from prostate cancer cell lines (LNCaP, DU 145, PC3, 22Rv1, VCaP), and 4 from other cell line types (HEK293T, Jurkat, HL-60, K562). Additionally, 11 samples were from the researcher themselves, with personal information anonymized. Specific primers targeting 5 SNP loci (rs2046210[C/T], rs2290203[C/T], rs11055880[C/T], rs4808611[C/T], and rs2236007[GA/CT]) were used in the PCR reactions. In the qPCR-based SNP genotyping analysis, the allele-specific Ct values obtained from qPCR were used to calculate the percentage content of each allele at the respective site in the sample, which determined the genotype. Taking rs4808611 as an example, the Ct values for the C allele-specific primer and T allele-specific primer were obtained from the qPCR reaction. The percentages of the two alleles were calculated using the formulas, the C allele ratio was calculated using the formula C %=2\hat{ }^{-Ct[C]}/2\hat{ }^{-Ct[C]}+2\hat{ }^{-Ct[T]}), and the T allele ratio was calculated using the formula T %=2\hat{ }^{-Ct[T]}/2\hat{ }^{-Ct[C]}+2\hat{ }^{-Ct[T]}). Additionally, the fluorescence values of the tested alleles were directly plotted as scatter plots to visually display the genotypes of the cell lines. The PCR conditions and program were described in the getPCR analysis conditions section. As a comparison, five commercial products were also used for genotyping at the rs2236007 site. These products include 2× Ultra SYBR Mix, THUNDERBIRD SYBR qPCR Mix, SYBR®Select Master Mix, Life Power, and 2×T5 Fast qPCR. The amplification conditions for each product were followed according to their respective product manuals.

1.8 PCR with Blocking Primers or LNA (Locked Nucleic Acid) Primers

Blocking primers or LNA primers with ddC or phosphate groups at the 3′ end can be used to enhance the selectivity of allele-specific amplification. Allele-specific primers, control amplification primers, and blocking primers targeting the homozygous TP53-G818A site in SW620 cell genomic DNA and the TP53-G839A site in MDA-MB-231 cell genomic DNA were designed to evaluate their ability to improve PCR selectivity. In a 15 μL qPCR reaction, we used 1× Taq buffer, 3 pmol of upstream and downstream primers, and 0.005 ng of PCR product containing the variant allele as the template. The PCR amplification program consisted of an initial denaturation at 95° C. for 5 minutes, followed by 45 cycles of 95° C. for 15 s, 68° C. for 15 s, and 72° C. for 15 s. Finally, a standard melting curve program was performed.

2. Results

2.1 Rational Design for Directed Evolution of Highly Selective Taq

Although large fragments lacking 5′ exonuclease activity (KlenTaq) could improve fidelity and thermal stability, a full-length Thermus aquaticus (Taq) DNA polymerase (SEQ ID NO: 1) was chosen as the starting molecule for molecular evolution to enable the final DNA polymerase variant to be compatible with both SYBR Green and TaqMan probe-based qPCR analyses. Researchers recognized that substituting amino acids that directly interact with the primer/template complex or affect the geometry of the binding pocket could alter the selectivity of the polymerase. In previous studies, researchers only selected a subset of amino acids that contact the primer/template for mutation. In the present invention, to identify candidate amino acids for rational design, the crystal structures of the open and closed forms of the DNA polymerase were investigated, and all 40 polar amino acids directly contacting the primer/template duplex were selected as targets for mutation (a of FIG. 1). Among these amino acids, 17 residues contact the primer, 24 residues contact the template, and 1 residue (Arg573) contacts both. For these selected amino acids, site-directed mutagenesis was performed, replacing the 40 polar amino acid residues with nonpolar residues such as leucine, alanine, or valine while attempting to maintain their spatial geometry. Specifically, amino acids N, R, Q, E, K, Y, D, M, and H were replaced with L, and S and T were replaced with A and V, respectively (see table below). Since the polar side chains of amino acids are typically directly involved in contacts, the substitution with nonpolar amino acid residues effectively disrupts the corresponding interactions, making the Taq polymerase more sensitive to primer/template mismatches and potentially improving its selectivity in mismatch extension.

The transformed colonies grown on LB agar plates containing IPTG were directly used for high-throughput screening without the need for complex protein purification procedures. Firstly, the activity and selectivity of the 40 Taq variants were evaluated using a colony qPCR system based on TaqMan probes. This screening system utilized 26 plasmids that simulated indels in the HOXB13 gene as templates. In this system, two amplicons were designed in a single reaction tube: one was a detection amplicon used to assess polymerase selectivity, where the detection primer could anneal to the wild-type DNA sequence, which is the region where indels occur in the genome; the other was a control amplicon used to evaluate polymerase activity and the primers annealed to a neighboring region (b of FIG. 1). The 26 indels would result in various mismatches with the detection primer, and an increase in the Ct value of the detection amplicon compared to wild-type Taq would indicate enhanced selectivity of the mutant variant. At the same time, if the Ct value of the control amplicon remained unchanged, it would indicate that the tested Taq mutant variant activity was not affected by the mutations.

It is found that nine variants had a severe loss with polymerase activity: R536L, Y545L, R573L, N580L, N583L, Y671L, N750L, Q754L, and H784L. Nineteen of the variations exhibited increased selectivity with statistical significance compared to the wild-type Taq, among which eight showed an increase of 5 cycles compared to wild-type Taq, indicating better selectivity (a of FIG. 2). However, even the variant T506V, which retained full activity and had the highest selectivity, could only improve by 13.9 cycles, indicating significant limitations.

| Item | |
|---|---|
| Number | Mutated amino acid |
| 1 | Taq-N483L |
| 2 | Taq-N485L |
| 3 | Taq-S486A |
| 4 | Taq-R487L |
| 5 | Taq-Q489L |
| 6 | Taq-T506V |
| 7 | Taq-E507L |
| 8 | Taq-K508L |
| 9 | Taq-T509V |
| 10 | Taq-S513A |
| 11 | Taq-T514V |
| 12 | Taq-S515A |
| 13 | Taq-R536L |
| 14 | Taq-K540L |
| 15 | Taq-S543A |
| 16 | Taq-T544V |
| 17 | Taq-Y545L |
| 18 | Taq-T569V |
| 19 | Taq-R573L |
| 20 | Taq-S575A |
| 21 | Taq-S576A |
| 22 | Taq-S577A |
| 23 | Taq-D578L |
| 24 | Taq-N580L |
| 25 | Taq-N583L |
| 26 | Taq-R587L |
| 27 | Taq-R660L |
| 28 | Taq-Q782L |
| 29 | Taq-H784L |
| 30 | Taq-T664V |
| 31 | Taq-Y671L |
| 32 | Taq-S674A |
| 33 | Taq-R677L |
| 34 | Taq-R728L |
| 35 | Taq-K738L |
| 36 | Taq-E742L |
| 37 | Taq-R746L |
| 38 | Taq-M747L |
| 39 | Taq-N750L |
| 40 | Taq-Q754L |

2.2 Molecular Evolution by Extensive Mutagenesis of High-Selectivity Taq Polymerase

Furthermore, extensive random mutagenesis was performed on these 40 variants and the wild-type Taq to screen for Taq variants with improved specificity. The GeneMorph II random mutagenesis kit was used to introduce minimal mutation bias and achieve a reasonable mutation rate. For directed protein evolution through random mutagenesis, typically each construct had 2-7 nucleotide mutations, corresponding to 1-3 amino acid mutations. By adjusting the input template amount and cycling times, a Taq mutant library with an average of 5.3 mutations in the Taq coding region was generated through error-prone PCR. The error-prone PCR products were then cloned into the prokaryotic expression plasmid pAKTaq, and the resulting single-cell colonies grown on LB agar plates containing IPTG were directly applied to the qPCR screening system for selection.

A total of 1,316 clones were screened (b of FIG. 2), of which 1,001 clones (76.1%) showed a rightward shift on the x-axis of the amplification curves and an increase of more than 5 cycles, indicating the loss of most or all of the polymerase activity. There were 101 clones (7.7%) that not only retained full activity but also exhibited high selectivity, with no amplification signal observed even for the amplification reactions detecting indel mismatches. To further confirm the specificity of these high-selectivity Taq variants, in addition to the 101 clones, an additional 75 clones were selected based on the criteria of Ct(Ctrl)<14.5 and Ct(Test)>30 (colored dots in c of FIG. 2). This time, lines were drawn on LB agar plates containing IPTG, colonies with diameters larger than 2 mm were collected, and they were evaluated in the qPCR screening system. It was found that only 62 colonies (35.2%) still met the criteria of Ct(Ctrl)<14.5 and Ct(Test)>30, which may reflect the poor stability of the colony qPCR system used earlier. Among these, 39 clones that met a higher standard (Ct(Ctrl)<14.5 and Ct(Test)>40) were selected for Sanger sequencing, and these Taq enzyme variants (listed in the table below) were expressed and purified in E. coli, and further validated using the purified Taq polymerase (represented by dots in c of FIG. 2). Interestingly, among these 39 variants, only 13 variants had amino acid substitutions that directly involved the interaction between Taq polymerase and the primer/template complex (a of FIG. 7).

| Variant | |
|---|---|
| identifiers | Mutated amino acid |
| Taq388 | S577A, W645R, I707V |
| Taq92 | R405Q, T569V |
| Taq99 | K354R, K531Q |
| Taq393 | L441M |
| Taq401 | S543A, R630W, F692Y, Y719F |
| Taq506 | M4I, D371E, V518D, A798V |
| Taq591 | G32D, D238V, W398C, N485L, I503F, R771K |
| Taq664 | E284K, I614L |
| Taq866 | T588S, L789F |
| Taq9 | G59W, V155F, K508Q |
| Taq1150 | R229G, E255V, Q489L |
| Taq1140 | E90K, E132Q, P369T, T513A |
| Taq761 | D151G, S515A, R741Q |
| Taq812 | A294S, A675V, E688D, V740A |
| Taq687 | G173D, L500I |
| Taq808 | R37Q, T140S, D365N |
| Taq1105 | T140A, L538I |
| Taq1151 | P10A, E303G, L484I, R492M |
| Taq1194 | F272S, E794D |
| Taq1108 | E170G, K508T, D578L, E818V |
| Taq1221 | I799F, K206R, R229W |
| Taq588 | R249C, V390M, E404G |
| Taq712 | E267V, S577A, Q680H |
| Taq1286 | R328M, R469C, |
| Taq1129 | E159D, D181H, P387L |
| Taq816 | A61T, D91N, K100E, K131N, A777V |
| Taq729 | P194H, P369T, T514V, Y719F |
| Taq1080 | A118S, R435W, E708D |
| Taq1312 | P6T, D177E, L252M, E465D, S699T |
| Taq1161 | E135V, P316S, G422W |
| Taq815 | T385A |
| Taq5 | R137C, P685S, E818K, L828V |
| Taq867 | A414T, S515A, A600T |
| Taq480 | S36I, E171K, S576A |
| Taq764 | E57D, D222Y |
| Taq926 | H28L, E112D |
| Taq903 | L245P |
| Taq1062 | R630L |
| Taq1201 | L351F, L657P, P816S |

2.3 Purification of Taq Variants and Validation of their Selectivity

As described above, the 39 Taq variants with improved specificity were expressed and purified in E. coli. The Taq variants exhibited similar purity in SDS-PAGE analysis, with an apparent molecular weight of 94 kDa (b of FIG. 7). The polymerase activity and selectivity of these variants in the qPCR screening system for indel detection were evaluated. Ultimately, 10 excellent variants were identified, which showed significantly improved selectivity compared to the wild-type Taq. These variants exhibited at least a 7-cycle increase in Ct values for detecting indel mismatches (P<0.05) (colored dots in d of FIG. 2). Among them, the Taq variant Taq388 showed the best selectivity, with an approximately 23-cycle increase. This variant was selected for further systematic evaluation and application in subsequent experiments.

Subsequently, the fidelity of the Taq388 variant in PCR amplification was evaluated through Sanger sequencing. The Taq coding sequence was amplified using Taq388 and cloned back into the original vector. After transformation into E. coli, single clones were picked and subjected to Sanger sequencing analysis to identify DNA mutations generated during PCR amplification. It was found that the fidelity of Taq388 was increased by 4.7-fold (c of FIG. 7). Importantly, the wild-type Taq exhibited three types of mutations, including 56.5% transitions, 39.1% transversions, and 4.4% deletions, while Taq388 only produced transition mutations (d of FIG. 7). In summary, multiple enhanced Taq enzyme variants were obtained, which exhibited significantly improved selectivity in amplifying primer/template mismatches caused by indels. Furthermore, the fidelity of Taq388 was increased by 4.7-fold in PCR amplification.

2.4 Enhanced Taq's Ability to Discriminate Mismatches

Subsequently, the discriminatory capabilities of the Taq388 variant were systematically evaluated for various types of primer/template mismatches. Firstly, the ability to discriminate indel mismatches was tested using a TaqMan probe-based qPCR screening system. The results demonstrated a 23-cycle increase in selectivity for Taq388 compared to the wild-type Taq polymerase, as observed during the screening process (a of FIG. 3). When the same primers and templates were tested using a SYBR Green-based qPCR system, Taq388 also exhibited significantly improved discrimination of indel mismatches, albeit to a lesser extent than the TaqMan probe-based system (b of FIG. 3). Furthermore, the variant's ability to recognize single nucleotide mismatches at the last or second-to-last position of the primers was systematically investigated. To generate single nucleotide mismatches, plasmids containing three types of single nucleotide variations at position HOXB13 c.251G were constructed as qPCR templates, including c.251G>A, c.251G>T, and c.251G>C (a and b of FIG. 4). By employing four primers with only a 3′ end nucleotide difference, analysis using SYBR Green-based qPCR revealed a substantial reduction in amplification signal from mismatched templates for Taq388 polymerase variant compared to the wild-type Taq in all 12 mismatch types (a of FIG. 4). Similarly, when using primers with different second-to-last nucleotides at the 3′ end, the Taq388 variant also exhibited higher selectivity in the presence of a mismatch at the second-to-last position of the primer's 3′ end (b of FIG. 4).

Next, the amplification selectivity of the Taq variant for single nucleotide mismatches was evaluated in practical applications using genomic DNA. qPCR analysis was performed on genomic DNA from MCF7 cells (c of FIG. 4) and T-47D cells (d of FIG. 4) with SNP genotypes C/C and T/T, respectively, using allele-specific primers targeting the rs4808611 site at the 3′ end. It was found that Taq388 variant exhibited higher selectivity compared to the wild-type Taq for both allele-specific primers. Specifically, for the T allele-specific primer, the Taq388 variant showed a reduction in off-target amplification intensity from C/C genotype genomic DNA of MCF7 cells by approximately 10 cycles (c of FIG. 4). Similarly, for the C allele-specific primer, the amplification level from T/T genotype genomic DNA of T-47D cells was reduced by over 10 cycles compared to Taq (d of FIG. 4). Additionally, similar results were observed at another SNP site, rs2236007. Specifically, for the A allele-specific primer, the Taq388 variant exhibited a reduction in amplification level from G/G genotype genomic DNA of T-47D cells by 10.5 cycles (a of FIG. 8), while for the G allele-specific primer, the amplification level from A/A genotype genomic DNA of VCaP cells was reduced by up to 7 cycles compared to Taq (b of FIG. 8).

In addition, the Taq388 variant was compared to five commercially available SYBR Green-based qPCR master mixes. It is noteworthy that Taq388 polymerase exhibited higher selectivity for primer/template mismatches caused by indels compared to all the listed commercial products (c of FIG. 8). Furthermore, the variant demonstrated superior selectivity in allele-specific PCR amplification of the rs2236007 site using genomic DNA samples with G/G and A/A genotypes compared to the commercial products (d of FIG. 8).

2.5 Application of Taq388 in Single-Cell Clone Genotyping for Genome Editing

In functional genomics research, it is often necessary to screen a large number of offspring individuals or single-cell clones after genome editing experiments to obtain experimental materials with the desired gene modifications. The use of an enhanced Taq polymerase with higher selectivity can greatly improve the accuracy of gene typing. Therefore, applying Taq388 to gene typing analysis of single clones, with a template of 26 plasmids used as templates in the screening system, was performed. In the TaqMan probe-based qPCR analysis using wild-type sequence-specific test primers, Taq388 showed significantly improved ability to discriminate insertions/deletions (indels) compared to wild-type Taq polymerase. On average, Taq388 exhibited a 16.9 cycle increase in discrimination for the 26 indel templates (a of FIG. 5), with 23 of the indel templates showing no amplification signal at all. This indicates that Taq388 has exceptional capabilities in recognizing and distinguishing primer/template mismatches caused by indels. In the SYBR Green-based qPCR analysis, Taq388 showed an average 10.7 cycle increase in discrimination between these 26 indels and the wild-type, demonstrating stronger amplification specificity compared to wild-type Taq (b of FIG. 5). Although not as remarkable as in the TaqMan probe-based qPCR analysis, the minimum Ct value difference between the wild-type construct and the insert-deletion construct in the SYBR Green-based qPCR analysis still exceeded 9 cycles, which is sufficient to accurately identify single-cell clones with insert-deletion sequences.

Next, the performance of Taq388 in gene typing analysis of 31 single-cell clones was evaluated using genomic DNA as a template in practical applications. These clones were generated through CRISPR/Cas9-mediated genome editing targeting the HOXB13 and DYRK1A genes in lenti-X 293T cells. Sanger sequencing revealed that 20 clones had biallelic indel mutations in the HOXB13 gene, and 11 single-cell clones had biallelic indel mutations in the DYRK1A gene. qPCR gene typing analysis showed that Taq388 exhibited superior ability to discriminate indel sequences from wild-type sequences, whether it was for gene editing in the HOXB13 gene or the DYRK1A gene (c and d of FIG. 5). For gene editing performed on the HOXB13 sgRNA target 2, Taq388 and wild-type Taq polymerase exhibited average ΔCt values of 14.2 and 10.1 cycles, respectively, in discriminating indels from wild-type sequences (c of FIG. 5). Specifically, when detecting the HT2-04 clone, wild-type Taq polymerase gave a ΔCt value of only 4 cycles, while Taq388 did not detect any significant amplification signal throughout all 45 PCR cycles. Regarding gene editing performed on the DYRK1A sgRNA target 1, Taq388 and wild-type Taq polymerase determined ΔCt values of 9.5 and 2.6 cycles, respectively, caused by indel mutations (d of FIG. 5). This indicates that the application of Taq388 can make genome editing detection more accurate and reliable.

2.6 Application of Taq388 in SNP Genotyping.

SNPs have many advantages as third-generation molecular markers, including widespread distribution and high genetic stability. They have been widely used in various fields such as molecular biology, disease prediction, and treatment. However, SNP detection largely relies on the nucleotide selectivity of DNA polymerase. Therefore, the potential application of Taq388 in SNP gene typing analysis was tested. In the test, 30 genomic DNA samples were used, including 19 samples from cell lines purchased from ATCC and 11 samples from the inventors. The samples were randomly shuffled and assigned numbers to conceal personal information. Taq388 was used for allele-specific SYBR Green qPCR amplification to perform gene typing analysis of five SNP sites: rs2236007, rs4808611, rs11055880, rs2290203, and rs2046210. The SNP genotypes of these 30 samples were determined by Sanger sequencing.

Two methods were used to determine the genotypes of the samples. Firstly, the allele-specific Ct values were used to calculate the proportion of each allele and determine the SNP genotypes, as described in FIG. 6. In theory, for a sample that is homozygous for allele 1, the calculated proportions of allele 1 and allele 2 should be 100% and 0%, respectively. For heterozygous samples, the percentages of the two alleles should fall between these two values. For the SNP site rs2236007, qPCR analysis using Taq388 showed accurate identification of the SNP genotypes for all samples. The A/A samples and G/G samples were located on the respective axes, and the G/A samples were positioned between them (a of FIG. 6). Unexpectedly, the 10 G/A samples were distributed in a relatively scattered region rather than concentrated around 50%. Examination of the corresponding Sanger sequencing chromatograms revealed a strong correlation between the proportions of alleles calculated by Taq388 qPCR gene typing and the relative peak heights in the Sanger sequencing chromatograms (a of FIG. 10). For example, the SK-BR-3 cell line had the highest proportion of allele A, which was consistent with the Sanger sequencing result showing a much higher peak for allele A compared to allele G. This indicates that the allele proportions calculated by Taq388 qPCR gene typing accurately reflect the genotype of the sample. In contrast, qPCR analysis using wild-type Taq polymerase resulted in all sample points clustering in the first quadrant, making it impossible to determine the genotypes of each sample (a of FIG. 6). Gene typing using Taq388 polymerase was also performed for the remaining four SNP sites: rs4808611 (b of FIG. 6), rs11055880 (c of FIG. 6), rs2290203 (d of FIG. 6), and rs2046210 (e of FIG. 6). The SNP genotypes of each sample were successfully determined. Furthermore, the dispersed distribution pattern of heterozygous genotypes exhibited a strong correlation with the corresponding peak heights in the Sanger sequencing chromatograms (b to e of FIG. 10).

The commonly used endpoint SNP genotyping technique utilizes TaqMan probes or allele-specific primers to distinguish different alleles. However, in the current situation, there is a pressing need to further improve the selectivity of PCR between alleles for accurate SNP genotyping. Therefore, the application of Taq388 in endpoint genotyping method was evaluated, which involves reading SYBR Green fluorescence after the allele-specific PCR amplification steps to determine the genotype of the sample. The analysis of the rs2236007 site demonstrated that, compared to wild-type Taq polymerase, Taq388 qPCR amplification was able to completely distinguish the three genotypes of G/G, G/A, and A/A (f of FIG. 6), while the samples with these three genotypes were clustered together and indistinguishable when using wild-type Taq qPCR amplification. Similarly, Taq388 polymerase was successfully used for genotyping of the other four SNP sites: rs4808611 (g of FIG. 6), rs11055880 (h of FIG. 6), rs2290203 (i of FIG. 6), and rs2046210 (j of FIG. 6).

In the present invention, a semi-rational directed evolution approach was used to improve the ability of full-length Taq polymerase to distinguish primer-template mismatches caused by genomic editing mutations during PCR amplification. Firstly, individual site-directed mutations were introduced to the 40 polar amino acids that directly interact with the primer/template duplex structure on Taq polymerase. Then, extensive random mutagenesis was performed on these variants as well as the wild-type Taq sequence to generate a comprehensive library of Taq mutants. Using a HOXB13 gene plasmid with indels as the PCR amplification template, several Taq variants with significantly improved specificity were selected through three rounds of screening and validation on a qPCR platform. Among these variants, Taq388 variant with S577A, W645R, and I707V substitutions showed the best performance. Taq388 variant exhibited extremely significant improvements in PCR selectivity for mismatches derived from indels and single nucleotide variations. In practical applications, this Taq variant significantly enhanced the accuracy of single-cell clone genotyping using the getPCR method and also made AS-qPCR SNP genotyping a more feasible approach.

All previous attempts to improve Taq nucleotide selectivity focused on discriminating single-nucleotide mismatches. However, the present invention is the first to specifically address the primer/template mismatches caused by genomic editing indels, using extensive directed evolution to obtain a better-performing Taq polymerase variant. Additionally, instead of using the commonly used Klenow fragment, the full-length Taq polymerase was used as the starting molecule in this invention. This allows the Taq388 variant to be applicable not only in SYBR Green-based qPCR but also in TaqMan probe-based qPCR applications.

The previous studies were mostly rational design, focusing on a portion of polar amino acid residues that contact the primer/template complex and their further combinational applications. Here, the present invention included all the 40 polar amino acid residues that directly contacted the primer/template duplex and further conducted random mutagenesis to generate a more comprehensive library. Notably, among the final 39 variants, only 13 variants involve amino acid substitutions at residues involved in the contact with the primer/template, and all of these selected improved variants contain amino acid mutations that do not participate in this contact. Furthermore, among the top 10 variants obtained, as many as 5 Taq variants have amino acid mutations that do not involve residues involved in the enzyme/primer/template interaction. These results indicate that replacing these primer/template non-contacting amino acids can also contribute to selectivity and provide a new direction for DNA polymerase evolution.

When applied in detecting genome editing variations, the Taq388 polymerase variant displayed a greatly enhanced ability to discriminate genetic modification from the wild-type sequence. This will enable getPCR to be more accurate and convenient for detecting genome editing efficiency and genotyping of single-cell clones. In terms of naturally occurring genetic variations, Taq388 also demonstrated excellent ability to discriminate SNP alleles in AS-qPCR analysis. Thanks to the excellent allele discrimination ability of Taq388 in PCR reactions, two simple but effective SNP genotyping methods have been achieved. One method is to calculate the allele ratio using allele-specific Ct values, and the other method is to generate endpoint fluorescence scatterplots of allele-specific PCR amplification. Both methods allow for easy and accurate identification of the three genotypes in the samples.

In summary, through semi-rational directed evolution, multiple variants of Taq polymerase have been developed that exhibit significantly improved selectivity for primer/template mismatches arising from genome editing indels. Among them, the best variant Taq388 has shown great potential in genome editing assays and genetic variation detection. The success of this strategy provides a new approach for the evolution of DNA polymerases.

Finally, it should be noted that the above are only preferred examples of the present invention and are not intended to limit the present invention. Although the present invention has been described in detail with reference to the foregoing examples, it will be apparent to those skilled in the art that various modifications and variations can be made to the technical solutions recorded in each example or that some technical features can be equivalently replaced. Any modifications, equivalent replacements, improvements, etc. made within the spirit and principle of the present invention shall fall within the protection scope of the present invention.

1. A variant of a Taq DNA polymerase, being mutated based on a wild-type Taq DNA polymerase having an sequence as shown in SEQ ID NO: 1, and comprises one or more mutation sites selected from a group consisting of S577A, W645R, 1707V, R405Q, T569V, K354R, K531Q, L441M, S543A, R630W, F692Y, Y719F, M41, D371E, V518D, A798V, G32D, D238V, W398C, N485L, 1503F, R771K, E284K, 1614L, T588S, L789F, G59W, V155F, K508Q, R229G, E255V, Q489L, E90K, E132Q, P369T, T513A, D151G, S515A, R741Q, A294S, A675V, E688D, V740A, G173D, L5001, R37Q, T140S, D365N, T140A, L5381, P10A, E303G, L4841, R492M, F272S, E794D, E170G, K508T, D578L, E818V, 1799F, K206R, R229W, R249C, V390M, E404G, E267V, S577A, Q680H, R328M, R469C, E159D, D181H, P387L, A61T, D91N, K100E, K131N, A777V, P194H, P369T, T514V, Y719F, A118S, R435W, E708D, P6T, D177E, L252M, E465D, S699T, E135V, P316S, G422W, T385A, R137C, P685S, E818K, L828V, A414T, S515A, A600T, 5361, E171K, S576A, E57D, D222Y, H28L, E112D, L245P, R630L, L351F, L657P and P816S; wherein the mutation sites are numbered based on the sequence shown in SEQ ID NO: 1.

2. The variant according to claim 1, comprising 1 to 6 mutation sites.

3. The variant according to claim 1, selecting from the following variants:

| variant | |
|---|---|
| identifiers | mutated amino acid |
| Taq388 | S577A, W645R, I707V |
| Taq92 | R405Q, T569V |
| Taq99 | K354R, K531Q |
| Taq393 | L441M |
| Taq401 | S543A, R630W, F692Y, Y719F |
| Taq506 | M4I, D371E, V518D, A798V |
| Taq591 | G32D, D238V, W398C, N485L, I503F, R771K |
| Taq664 | E284K, I614L |
| Taq866 | T588S, L789F |
| Taq9 | G59W, V155F, K508Q |
| Taq1150 | R229G, E255V, Q489L |
| Taq1140 | E90K, E132Q, P369T, T513A |
| Taq761 | D151G, S515A, R741Q |
| Taq812 | A294S, A675V, E688D, V740A |
| Taq687 | G173D, L500I |
| Taq808 | R37Q, T140S, D365N |
| Taq1105 | T140A, L538I |
| Taq1151 | P10A, E303G, L484I, R492M |
| Taq1194 | F272S, E794D |
| Taq1108 | E170G, K508T, D578L, E818V |
| Taq1221 | I799F, K206R, R229W |
| Taq588 | R249C, V390M, E404G |
| Taq712 | E267V, S577A, Q680H |
| Taq1286 | R328M, R469C, |
| Taq1129 | E159D, D181H, P387L |
| Taq816 | A61T, D91N, K100E, K131N, A777V |
| Taq729 | P194H, P369T, T514V, Y719F |
| Taq1080 | A118S, R435W, E708D |
| Taq1312 | P6T, D177E, L252M, E465D, S699T |
| Taq1161 | E135V, P316S, G422W |
| Taq815 | T385A |
| Taq5 | R137C, P685S, E818K, L828V |
| Taq867 | A414T, S515A, A600T |
| Taq480 | S36I, E171K, S576A |
| Taq764 | E57D, D222Y |
| Taq926 | H28L, E112D |
| Taq903 | L245P |
| Taq1062 | R630L |
| Taq1201 | L351F, L657P, P816S |

4. A polynucleotide molecule, encoding a variant of a Taq DNA polymerase according to claim 1.

5. A recombinant expression vector, comprising a polynucleotide molecule, wherein the polynucleotide molecule encoding a variant of a Taq DNA polymerase according to claim 1.

6. A host cell, comprising a recombinant expression vector or having a polynucleotide molecule chromosomally integrated, wherein the recombinant expression vector comprising a polynucleotide molecule, and the polynucleotide molecule encoding a variant of a Taq DNA polymerase according to claim 1.

7. The host cell according to claim 6, wherein the host cell is prokaryotic cell or eukaryotic cell.

8. A method for preparing a variant of a Taq DNA polymerase according to claim 1, comprising: culturing a host cell, thereby expressing the variant; and isolating the variant.

9. A kit, comprising a variant of a Taq DNA polymerase according to claim 1.

10. An application of a variant of a Taq DNA polymerase according to claim 1, a polynucleotide molecule encoding the variant, a recombinant expression vector comprising the polynucleotide molecule, a host cell comprising the recombinant expression vector or having the polynucleotide molecule chromosomally integrated, or a kit comprising the variant in genome editing detection and/or gene mutation detection.
